(19) United States
(12) Patent Application Publication
(10) Pub. No.: US 2022/0175110 A1
(43) Pub. Date: Jun. 9, 2022

(54) DUO BOTTLE PACK FOR TOPICAL COSMETIC NAIL REPAIR AND ANTIFUNGAL TREATMENT

(71) Applicant: Kramer Laboratories, Inc., Bridgewater, NJ (US)

(72) Inventors: Sathappan C. Chinnakaruppan, Macungie, PA (US); Evan Bashoff, Summit, NJ (US)

(21) Appl. No.: 17/115,915

(22) Filed: Dec. 9, 2020

(51) Int. Cl. A45D 34/06; A45D 34/04

(52) U.S. Cl. CPC A45D 34/06; A45D 2200/1009; A45D 34/046; A45D 34/043

(57) ABSTRACT

Disclosed herein is an apparatus including two containers assembled together in a sleeve, where the first container includes a cosmetic agent, such a nail whitening agent, and the second container includes an antifungal active pharmaceutical ingredient. Also disclosed herein are methods for assembling an apparatus for treatment of nail fungus and a method of treating nail and/or skin fungus.

### FIELD OF THE INVENTION

The present invention relates generally to a duo bottle pack (a topical cosmetic nail formula and a topical antifungal skin treatment), methods of preparation of the duo bottle pack, and methods of use of the duo bottle pack.

### BACKGROUND OF THE INVENTION

Feet exposed to a warm, dark, moist environment are more prone to get infected with skin and nail fungus. For those who are susceptible, the condition can be highly contagious. The best way to avoid fungus is to keep areas that are more susceptible from getting infected, such as keeping feet clean and dry and using topical products that are approved to prevent skin fungus, since the skin can harbor fungus that can infect nails.

Nail fungus is a common condition that frequently occurs with skin fungus such as athlete's foot. Nail fungus causes the nail to discolor (e.g., exhibit yellow-brown discoloration), thicken, loosen, become brittle, harden, and sometimes crumble. If left untreated, it can spread from one area to another and ultimately affect different parts of the nail as well as spread to other nails and to the skin around the nails, between the toes, and other parts of the body. In addition to its unpleasant appearance, nail fungus can impact the quality of life of individuals suffering from it.

Current nail fungus treatment options include cosmetics, drug products (over the counter (OTC) in some countries; prescription in other countries), and medical devices. Examples of treatment options include, without limitations, topical creams, nail lacquers, topical prescriptions, oral prescription pills, laser treatment that removes the damaged area of the nail or skin, surgery and others. Current skin fungus treatment options include OTC and prescription antifungal products and oral prescription antifungals. Some drug products are approved to treat fungal infections and others approved to treat and prevent fungal infections.

There remains a need for novel and unique treatment options that provide nail improvement and skin fungus treatment or prevention in one packaging for a compact, safe, quick, and effective all-in-one solution.

### SUMMARY OF THE INVENTION

In some embodiments, the present invention relates to a duo-bottle pack apparatus that includes a first container having a cosmetic nail whitening composition and a second container having a topical skin anti-fungal composition.

In some embodiments, the duo-bottle pack apparatus includes a first container having a first bottom closed end, a first top open end opposite from the first bottom closed end, and a first wall extending from the first bottom closed end to the first top open end. The apparatus further includes a first cap coupled to a first applicator, where the first cap is configured to removably close the first top open end of the first container. The apparatus further includes a second container having a second bottom closed end, a second top open end opposite from the second bottom closed end, and a second wall extending from the second bottom closed end to the second top open end. The apparatus further includes a second cap coupled to a second applicator, where the second cap is configured to removably close the second top open end of the second container. The apparatus further includes a sleeve extending along the first wall and the second wall. The first container and the second container are assembled in the common sleeve such that the first bottom closed end is proximate to the second bottom closed end and the first top open end faces an opposite direction from the second top open end.

In some embodiments, the present invention relates to a method for preparing any of the dual bottle pack apparatuses disclosed herein by assembling a first container having a nail whitening composition and a second container having a topical skin antifungal composition in a sleeve.

In some embodiments, the present invention relates to a method of treating or improving the look of fungal nails. The method includes applying a cosmetic nail whitening composition from a first container, containing the nail whitening composition, on the target area (e.g., onto the nail) and applying a topical drug antifungal composition from a second container, containing the antifungal composition, onto a target area (e.g., onto the skin surrounding the nail and between the toes), wherein the first container and the second container are part of the same apparatus and are assembled together in one apparatus. In some embodiments, the method includes applying the nail whitening composition onto the skin surrounding the nail and/or between the toes with a first applicator and applying an anti-fungal composition onto the nail with a second applicator. The first container, second container, first applicator, and/or second applicator may be part of any of the duo bottle pack apparatuses described herein.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 depicts breakdown of various components of a duo bottle pack apparatus according to embodiments described herein;

FIG. 2 depicts a cross-sectional view of a duo bottle pack apparatus in a closed configuration according to embodiments described herein;

FIG. 3 depicts a perspective view of a closed duo bottle pack apparatus according to embodiments described herein, the duo bottle pack apparatus having circumferential symmetry;

FIG. 4 is a top plan view of a closed duo bottle pack apparatus according to embodiments described herein;

FIG. 5 is a bottom plan view of a closed duo bottle pack apparatus according to embodiments described herein;

FIG. 6 is an exploded perspective view of a duo bottle pack apparatus according to embodiments described herein, showing certain components of the duo bottle pack apparatus;

FIG. 7 is a partially exploded upper perspective view, showing components of the upper portion of the duo bottle pack apparatus from one angle;

FIG. 8 is a partially exploded bottom perspective view, showing components of the upper portion of the duo bottle pack apparatus from a different angle;

FIG. 9 is a partially exploded upper perspective view, showing components of the bottom portion of the duo bottle pack apparatus from one angle; and

FIG. 10 is a partially exploded bottom perspective view, showing components of the bottom portion of the duo bottle pack apparatus from a different angle.

FIG. 11 is a perspective view of bottle stopper according to an embodiment.

### DETAILED DESCRIPTION

As used herein, the singular forms “a,” “an,” and “the” include plural references unless the context clearly indicates otherwise. Thus, for example, reference to “a material” includes a single material as well as a mixture of two or more different materials; and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

Recitation of ranges of values herein are merely intended to serve as a shorthand method of referring individually to each separate value falling within the range, unless otherwise indicated herein, and each separate value is incorporated into the specification as if it were individually recited herein. Reference throughout this specification to numerical ranges should not be construed as limiting and should be understood as encompassing the outer limits of the range as well as each number and/or narrower range within the enumerated numerical range.

All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

Duo Bottle Pack Apparatus

In some embodiments, the instant disclosure is directed to a dual bottle pack apparatus that includes a first container and a second container assembled together in a sleeve. In one embodiment, the first container has an anti-fungal composition and the second container has a cosmetic agent composition such as a nail whitening composition. In another embodiment, the first container has a cosmetic agent composition such as a nail whitening composition and the second container has an anti-fungal composition. While the instant description may refer to the contents of the first container and the second container as including an anti-fungal composition and a cosmetic agent composition, such reference should not be construed as limiting. It should be understood by those skilled in the art that other compositions may be suitably filled in the first container and second container and that the duo bottle pack apparatus described herein may be useful for other uses besides anti-fungal treatment.

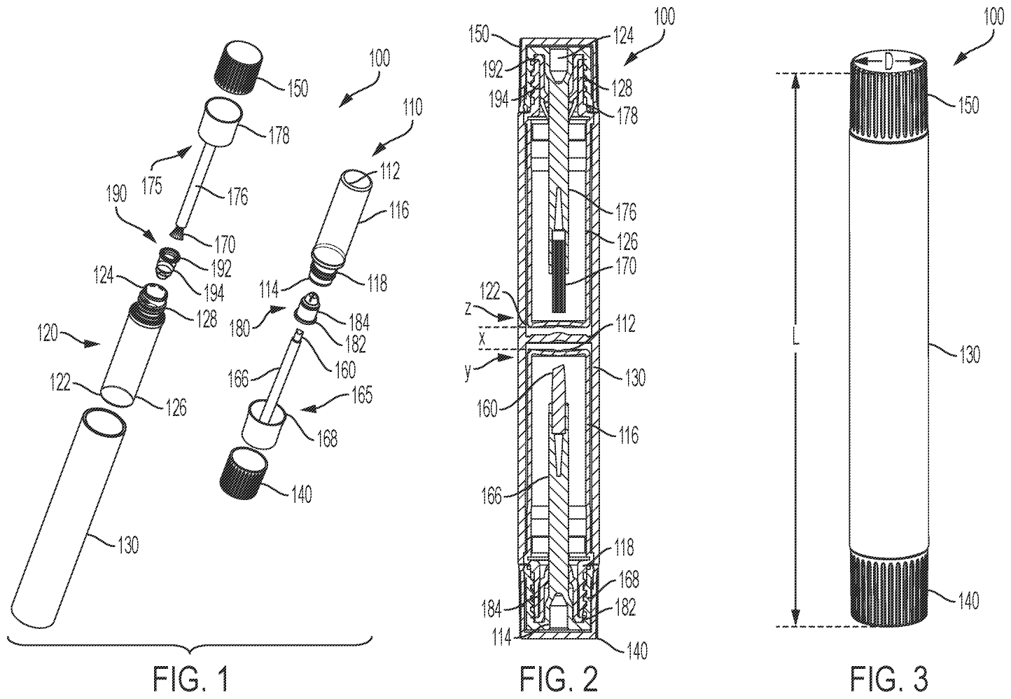

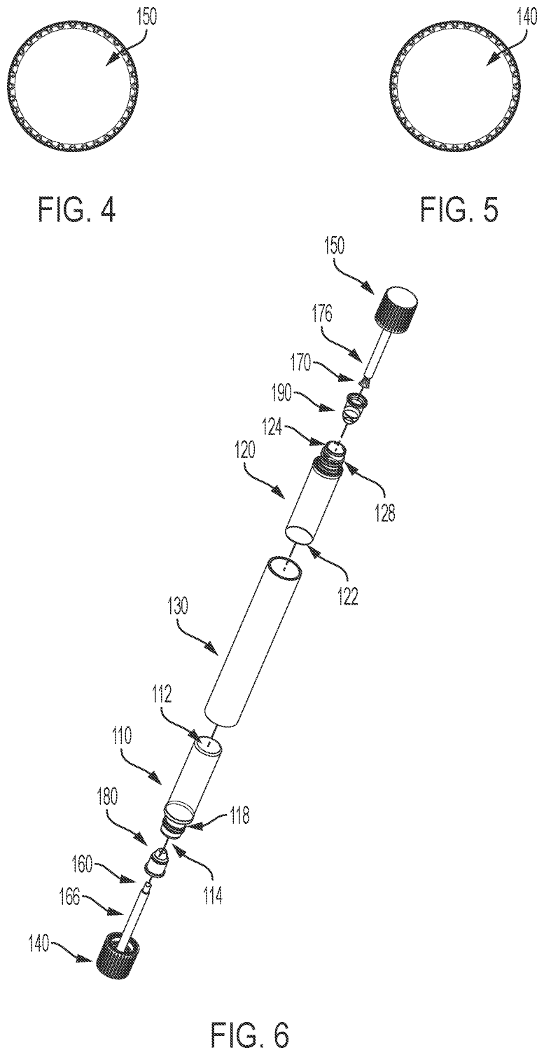

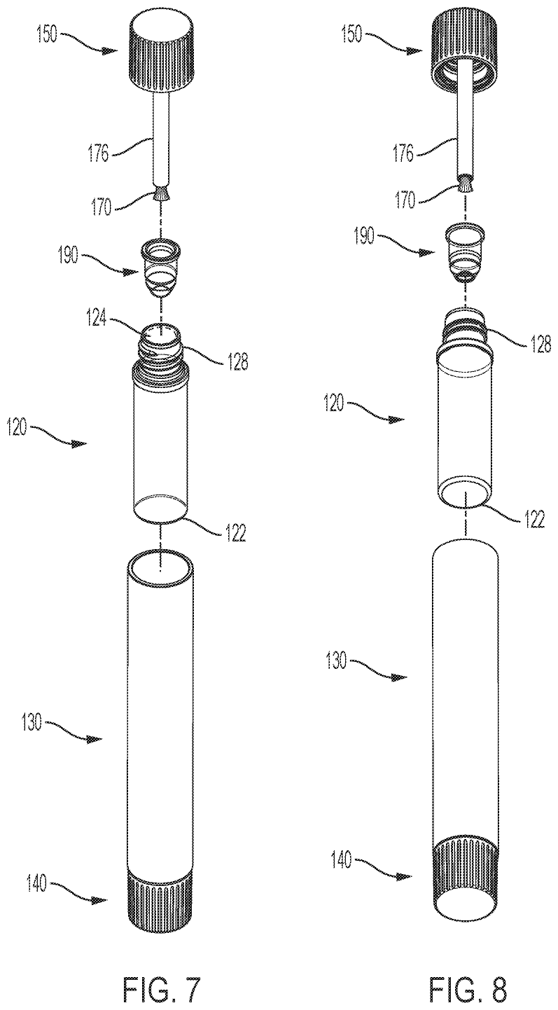

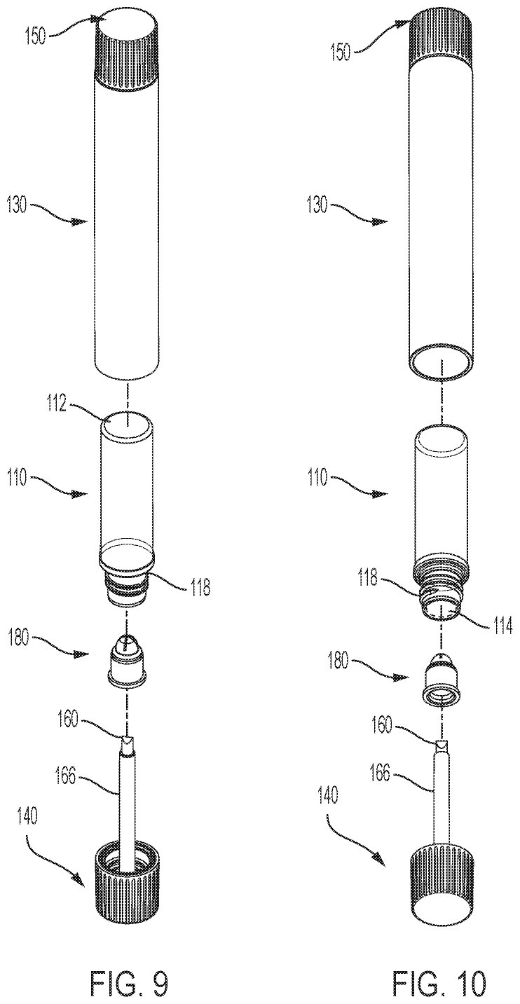

Turning to the figures, a duo bottle pack apparatus according to embodiments of the disclosure will be described in more detail. FIG. 1 depicts breakdown of various components of a duo bottle pack apparatus 100 according to embodiments described herein. FIG. 2 depicts a cross-sectional view of duo bottle pack apparatus 100 in a closed configuration according to embodiments described herein. FIG. 3 depicts a perspective view of duo bottle pack apparatus 100 in a closed configuration according to embodiments described herein, the duo bottle pack apparatus having circumferential symmetry. FIG. 4 is a top plan view of a closed duo bottle pack apparatus according to embodiments described herein. FIG. 5 is a bottom plan view of a closed duo bottle pack apparatus according to embodiments described herein. FIG. 6 is an exploded perspective view of a duo bottle pack apparatus according to embodiments described herein, showing certain components of the duo bottle pack apparatus. FIG. 7 is a partially exploded upper perspective view, showing components of the upper portion of the duo bottle pack apparatus from one angle. FIG. 8 is a partially exploded bottom perspective view, showing components of the upper portion of the duo bottle pack apparatus from a different angle. FIG. 9 is a partially exploded upper perspective view, showing components of the bottom portion of the duo bottle pack apparatus from one angle. FIG. 10 is a partially exploded bottom perspective view, showing components of the bottom portion of the duo bottle pack apparatus from a different angle.

In certain embodiments, duo bottle pack apparatus 100 includes a first container 110 and a second container 120 assembled in a common sleeve 130. In the embodiments shown, the first container 110 includes a first bottom closed end 112, a first top open end 114 opposite from the first bottom closed end, and a first wall 116 extending from the first bottom closed end 112 to the first top open end 114. Similarly, the second container 120 includes a second bottom closed end 122, a second top open end 124 opposite from the second bottom closed end, and a second wall 126 extending from the second bottom closed end 122 to the second top open end 124. In the embodiments shown (as best seen in FIG. 2), the first container 110 and the second container 120 are assembled in the sleeve such that the first bottom closed end 112 is proximate to the second bottom closed end 122 and the first top open end 114 faces an opposite direction from the second top open end 124. While the containers depicted in the figures are rounded tubular bottles, the shape of the container should not be construed as limited to the depicted embodiment. Any other suitable container shape may be utilized herein.

In the embodiments shown, the sleeve 130 extends along the first wall 116 and the second wall 126. The sleeve 130 may extend along the entirety of the first wall 116 and the second wall 126, as shown in FIGS. 2 and 3. Alternatively, sleeve 130 may extend along a portion of the first wall 116 and the second wall 126. In certain embodiments, the duo bottle pack has circumfrential symmetry, i.e., where all sides of the duo bottle pack (in a closed configuration) are symmetricaly the same.

In certain embodiments, first container 110 and second container 120 may be assembled inside sleeve 130 removably or non-removably. For instance, sleeve 130 may be glued to the first container 110 and the second container 120. For example, sleeve 130 may be glued to first wall 116 of first container 110 and to second wall 126 of second container 120, such that the first container 110 and the second container 120 remain in place and are not removable from sleeve 130. Alternatively, sleeve 130 may wrap around, slip onto, or be mechanically coupled (e.g., through notches or other mechanical means) to the first container 110 and the second container 120 so as to be removable.

In certain embodiments, the first closed bottom end 112 of first container 110 may be spaced from the second closed bottom end 122 of the second container by space x. Space x may range from any of 0 mm, about 0.5 mm, about 1.0 mm, about 1.5 mm, about 2.0 mm, about 2.5 mm, about 3.0 mm to any of about 3.5 mm, about 4.0 mm, about 4.5 mm, about 5.0 mm, about 6.0 mm, about 7.0 mm, about 8.0 mm, about 9.0 mm, about 10.0 mm, about 15.0 mm, or about 20.0 mm, or any sub-range or single value therein. In one embodiment, space x is 0 such that the first closed bottom end 112 is in contact with the second closed bottom end 122.

In certain embodiments, first container 110 and second container 120 may be assembled inside sleeve 130 and/or aligned within sleeve 130 and/or connected to each other removably or non-removably. For instance, first closed bottom end 112 may be glued to the second closed bottom end 122 such that the first container 110 and the second container 120 remain connected to each other non-removably. Alternatively, first closed bottom end 112 and second closed bottom end 122 may be mechanically coupled to each other or to an intermediate component within sleeve 130 so as to be removable. For instance, in one embodiment, one of the containers may have a “gate” or a dome shaped feature on its corresponding bottom closed end and the other container may have a cavity shaped feature on its corresponding bottom closed end so that the “gate” or dome shaped feature on one container engages the cavity shaped feature on the other container, thereby connecting and/or aligning the containers in the sleeve.

In the depicted embodiments, the top open end of each container includes a corresponding neck portion that transitions into the corresponding wall of each container. Thus, the first top open end 114 includes a first neck portion 118 that transitions into first wall 116. Similarly, the second top open end 124 includes a second neck portion 128 that transitions into second wall 126.

In certain embodiments, neck portion 118 engages a first cap 140 to removably close the first top open end 114 of the first container. Similarly, in certain embodiments, neck portion 128 engages a second cap 150 to removably close the second top open end 124 of the second container. First cap 140 and second cap 150 may be screw caps that screw onto threads of first neck portion 118 and second neck portion 128, respectively. The top and the bottom views of caps 140 and 150 are shown in FIGS. 4 and 5. Any other suitable cap may be utilized to close the first container and the second container. For instance, the cap may include, without limitations, a click lock feature, an orientation feature, a snap cap, a hinged cap, a flange cap, a plug, or any other suitable cap. The term “removably,” as used herein, refers to the possibility of manipulating the cap to provide access to the top open end of each container (either directly or through a cap rod assembly) and does not require that the cap be completely removed from the container. In one embodiment, a removable cap may refer to a screw cap that is configured to be completely removed from the container to open the container. In another embodiment, a removable cap may refer to a hinged cap that is configured to stay affixed to the container by a hinge upon opening the container.

The caps may further include a variety of outer surface textures. For instance, in the depicted embodiment, the outer surface of the first cap 140 and second cap 150 is ribbed. Such outer surface textures may provide a better grip for an individual when opening each container. In other embodiments, the outer surface of the caps may be smooth. The outer surface of the first cap 140 and of the second cap 150 may be the same or different.

In certain embodiments, each cap is coupled to a corresponding applicator, optionally via a corresponding cap rod assembly. In the depicted embodiments, first cap 140 is coupled to a first applicator 160 via a first cap rod assembly 165. Similarly, second cap 150 is coupled to a second applicator 170 via a second cap rod assembly 175. The cap rod assemblies are shown in their entirety in FIG. 1.

In certain embodiments, each cap rod assembly includes a stem coupled to an interior cap assembly. Each interior cap assembly is configured to couple each cap, through the cap rod (and correspondingly through the stem) to each applicator. The interior cap assembly may be coupled to the cap removably or non-removably. In FIGS. 6-10, the interior cap assemblies are inserted into the corresponding cap and only the stem portion of the cap rod assembly is shown.

In the depicted embodiments, first cap rod assembly 165 includes a first stem 166 and a first interior cap assembly 168. Similarly, second cap rod assembly 175 includes a second stem 176 and a second interior cap assembly 178. In the depicted embodiment, first interior cap assembly 168 and second interior cap assembly 178 include a screw cap feature configured to engage first neck portion 118 and second neck portion 128, respectively, to close the first container 110 and the second container 120, respectively. It should be noted that the depicted embodiment is merely exemplary and in certain embodiments a different interior cap assembly may be utilized. In certain embodiment, the cap rod may be comprised of a stem that may be directly coupled to the cap of the container (e.g., cap 140 and/or cap 150) without an additional interior cap assembly.

A variety of applicators may be utilized with the apparatus described herein. In certain embodiments, the first applicator 160 and/or the second applicator 170 may include a brush tip of a brushless tip. Examples of suitable applicators include, without limitations, a felt tip, a sponge, a brush. The tip of the first applicator and the second application, whether a brush tip or a brushless tip, may have any shape suitable for applying the contents of the first container and the second container, respectively. For instance, the tip of the first applicator and the second application, may have a shape independently selected from round, flat, domed, fan, angled, or any other suitable shape. In the depicted embodiment, first applicator 160 is an angled felt tip and the second applicator 170 is a flat brush tip. In one embodiment, the felt tip may also be of a pointed shape.

The length of each applicator may also be variable. In certain embodiments, the length of first applicator 160 and second applicator 170 independently ranges from any of about 0.5 mm, about 1.0 mm, about 1.5 mm, about 2.0 mm, or about 2.5 mm to any of about 3.0 mm, about 3.5 mm, about 4.0 mm, about 4.5 mm, or about 5.0 mm, or any sub-range or single value therein. In one embodiment, the length of the first applicator 160 and the second applicator 170 ranges from about 1.0 mm to about 5.0 mm. The length of the first applicator 160 may be the same or different from the length of the second applicator 170.

Each container includes an interior volume that may be filled with a composition (e.g., an antifungal composition or a cosmetic composition). In the depicted embodiment, the first bottom closed end 112, the first top open end 114 (along with the first neck portion 118), and the first wall 116 together define a first interior of the first container 110. Similarly, the second bottom closed end 122, the second top open end 124 (along with the second neck portion 128), and the second wall 126 together define a second interior of the second container 120. In certain embodiments, the first interior of the first container 110 is filled with an active pharmaceutical ingredient, such as an anti-fungal composition, and the second interior of the second container 120 is filled with a cosmetic agent, such as a nail whitening composition. In alternative embodiments, the first interior of the first container 110 is filled with a cosmetic agent, such as a nail whitening composition and the second interior of the second container 120 is filled with an active pharmaceutical ingredient, such as an anti-fungal composition.

The fill composition and/or the applicator type should not be construed as limited to the embodiments depicted herein, so long as each fill composition applied with the proper applicator for optimal administration of the fill composition onto the target surface. For instance, a felt tip, such as that of first applicator 160, may be suitable for optimal administration of a cosmetic agent, such as a nail whitening composition, to a targeted area on the nail, such as the nail bed surface. Similarly, a flat brush tip, such as that of second applicator 170, may be suitable for optimal administration of an anti-fungal composition onto the nail bed surface, the skin around the nail, or the skin between the toes. In such an embodiment, the fill composition of the first container 110 (equipped with a felt tip first applicator 160) will be a cosmetic nail whitening composition or nail whitening medical device and the fill composition of the second container 120 (equipped with a flat brush tip second applicator 170) will be an anti-fungal composition.

In certain embodiments, each cap rod assembly may further include a wiper situated between the circumference of the top open end (or of the neck portion) of each container and the stem of the cap rod. The wiper is utilized to wipe off excess material extracted from the interior of each container to control the amount extracted and to provide for a mess free application of the composition onto the target surface (e.g., onto the nail or the skin surrounding the nail). In the depicted embodiment, the first wiper 180 has hollow tubular shape defined by a first tubular wall portion 184 and a first lip portion 182 that is wider (e.g., has a larger diameter) than the first tubular wall portion 184. The first tubular wall portion 184 of the first wiper 180 is situated in the first neck portion 118 (on the interior side) in the assembled configuration of the apparatus. The first lip portion 182 of the first wiper 180 sits on the circumference of the first top open end 114 in the assembled configuration of the apparatus. Similarly, the second wiper 190 has a hollow tubular shape defined by a second tubular wall portion 194 and a second lip portion 192 that is wider (e.g., has a larger diameter) than the second tubular wall portion 194. The second wall portion 194 of the second wiper 190 is situated in the second neck portion 128 (on the interior side) in the assembled configuration of the apparatus. The second lip portion 192 of the second wiper 190 sits on the circumference of the second top open end 124 in the assembled configuration of the apparatus.

Each container can be independently closed or opened. In certain embodiments, each container may be closed airtight such that substantially no air can penetrate the container. An exemplary configuration of an assembled apparatus when both containers are closed is depicted in FIGS. 2 and 3 and described in further detail below. FIGS. 7-8 further depict an exemplary configuration, from two different angles, of a partially assembled apparatus, where the bottom container is closed and the components of the top portion are shown. FIGS. 9-10 further depict an exemplary configuration, from two different angles, of a partially assembled apparatus, where the top container is closed and the components of the bottom portion are shown.

First cap rod assembly 165, which includes first interior cap assembly 168 and first stem 166, coupled to first applicator 160, is inserted into the first interior of the first container 110, with the first interior cap assembly 168 engaging the first neck portion 118 to close the first container. The first stem 166 of the first cap rod assembly 165 and the first brush applicator 160 are inserted through an opening in the first tubular wiper 180 positioned over the first top open end 114 (i.e., the first lip portion 182 of the first wiper 180) and within the interior of the first neck portion 118 (i.e., the first tubular wall portion 184 of the first wiper) of the first container 110.

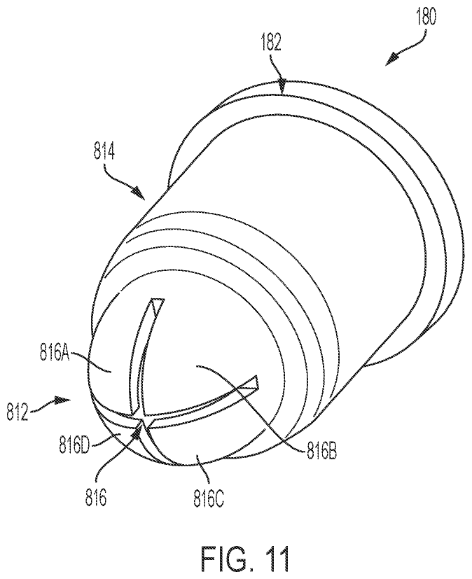

In certain embodiments, wiper 180 may have a dome shape, e.g., as shown in FIG. 11. The dome shape may have a semi-spherical shape on one end 812, transition to a cylindrical portion 814 (with a diameter that is optionally greater than the diameters of the semi-spherical portion on end 812), and a lip portion 182. Wiper 180 may act as a rubber stopper or a bottle stopper configured to make the first container spill proof or to minimize spills or leaks from the first container even when the first container is inverted (i.e. facing downwards) and the contents of the first container have a low viscosity (e.g., an easily flowing liquid). For instance, wiper 180, which may also be referred to as a bottle stopper, may have a slit 816 (e.g., a “X” or cross type slit) in the semi-spherical shape on the one end 812 (the side that is opposite to the lip portion 182). The slit 816 in the wiper 180 may be configured to provide an opening for the first stem 166 to be inserted therethrough such that the slit 816 (e.g., the four quadrants of the slit 816A, 816B, 816C, and 816D) expands when the first stem 166 penetrates therethrough. The slit 816 in the wiper 180 may engage the first stem 166 and surround it tightly so as to minimize spillage or leakage of the contents of container 110 when the first stem 166 penetrates through the slit 816. When first stem 166 (or the first applicator 160) does not penetrate through the slit 816 in wiper 180, the slit may shut tightly so that the contents of container 110 do not spill out (for instance, if a user forgets to cap the first container and inverts it without the cap, the wiper 180 seals the first container, thereby minimizing or eliminating spills and/or leaks). In certain embodiments, a wiper 180 having the slit configuration, as described herein, contributes to formation of a leak proof (or spill proof) bottom container.

The first cap 140 closes the first container, by engaging at least one of the first cap rod assembly 165 (e.g., via the first interior cap assembly 168), the first wall 116, the first top open end 114, the first neck portion 118, the sleeve 130, or the first wiper 180 (e.g., via the first lip portion 182). The first cap 140 may close the first container 110 in an air tight manner. In one embodiment, the first cap 140 closes the first container via a click lock mechanism. In one embodiment, the first cap 140 closes the first container via a screw on mechanism.

In the closed configuration, the tip of the first applicator 160 may reach the first closed bottom end 112 of the first container 110 or may be spaced away by distance y from the first closed bottom end 112 of the first container 110. For instance, distance y may range from any of 0 mm, about 0.5 mm, about 1.0 mm, about 1.5 mm, about 2.0 mm, about 2.5 mm, about 3.0 mm to any of about 3.5 mm, about 4.0 mm, about 4.5 mm, about 5.0 mm, about 6.0 mm, about 7.0 mm, about 8.0 mm, about 9.0 mm, or about 10.0 mm. In one embodiment, distance y is about 1.0 mm to about 5.0 mm.

Second cap rod assembly 175, which includes second interior cap assembly 178 and second stem 176, coupled to second applicator 170, is inserted into the second interior of the second container 120, with the second interior cap assembly 178 engaging the second neck portion 128 to close the second container. The second stem 176 of the second cap rod assembly 175 and the second applicator 170 are inserted through an opening in the second tubular wiper 190, positioned over the second top open end 124 (i.e., the second lip portion 192 of the second wiper 190) and within the interior of the second neck portion 128 (i.e., the second tubular wall portion 194 of the second wiper 190) of the second container 120. The second cap 150 closes the second container, by engaging at least one of the second cap rod assembly 175 (e.g., via the second interior cap assembly 178), the second wall 126, the second top open end 124, the second neck portion 128, the sleeve 130, or the second wiper 190 (e.g., via the second lip portion 192). The second cap 150 may close the second container 120 in an air tight manner. In one embodiment, the second cap 150 closes the second container 120 via a click lock mechanism. In one embodiment, the second cap 150 closes the second container 120 via a screw on mechanism.

In the closed configuration, the tip of the second applicator 170 may reach the second closed bottom end 122 of the second container 120 or may be spaced away by distance z from the second closed bottom end 122 of the second container 120. For instance, distance z may range from any of 0 mm, about 0.5 mm, about 1.0 mm, about 1.5 mm, about 2.0 mm, about 2.5 mm, about 3.0 mm to any of about 3.5 mm, about 4.0 mm, about 4.5 mm, about 5.0 mm, about 6.0 mm, about 7.0 mm, about 8.0 mm, about 9.0 mm, or about 10.0 mm. In one embodiment, distance z is about 1.0 mm to about 5.0 mm.

The assembled apparatus may be compact enough to be conveniently stored and/or transported without taking much space. In certain embodiments, the assembled apparatus may have a length, L, ranging from any of about 20 mm, about 30 mm, about 40 mm, about 50 mm, about 60 mm, about 70 mm, about 80 mm, about 90 mm, or about 100 mm to any of about 110 mm, about 120 mm, about 130 mm, about 140 mm, about 150 mm, about 160 mm, about 170 mm, about 180 mm, about 190 mm, or about 200 mm, or any sub-range or single value therein. In one embodiment, L range from about 130 mm to about 150 mm.

In certain embodiments, the length first cap and the second cap, together, account for up to about 5%, up to about 10%, up to about 15%, up to about 20%, up to about 25%, up to about 30%, up to about 40%, or up to about 50%, or any sub-range or single value therein, of the length L of the assembled apparatus. For instance, in one embodiment, the length of the first cap and the second cap, together, accounts for about 25% of the length L of the assembled apparatus and ranges from about 30 mm to about 40 mm. In certain embodiments where the first cap and the second cap are the same, their length may also be the same. Alternatively, the first cap may differ from the second cap and each may have different dimensions.

In certain embodiments, the length of the sleeve accounts for about 100%, about 95%, about 90%, about 85%, about 80%, about 75%, about 70%, about 60%, or about 50%, or any sub-range or single value therein, of the length L of the assembled apparatus. For instance, in one embodiment, the length of the sleeve accounts for about 75% of the length L of the assembled apparatus and ranges from about 100 mm to about 110 mm.

In certain embodiments, the assembled apparatus may have a width D (e.g., diameter where the apparatus is tubular) ranging from any of about 5 mm, about 10 mm, or about 15 mm to any of about 20 mm, about 25 mm, or about 30 mm, or any sub-range or single value therein. In one embodiment, D ranges from about 15 mm to about 20 mm.

In certain embodiments, the width of the container opening (e.g., the width of the wiper orifice in the depicted embodiment) may account for about 5%, about 10%, about 15%, about 20%, about 25%, about 30%, about 40%, or about 50%, or any sub-range or single value therein, of the width D of the apparatus. In one embodiment, the width of the wiper orifice accounts for about 25% of D and ranges from about 3.5 mm to about 5.0 mm.

In certain embodiments, the depth of each container, defined from the top open end of each container (through the neck portion and wall portion) to the bottom closed end of each container, ranges, independently, from any of about 10 mm, about 15 mm, about 20 mm, about 25 mm, about 30 mm, about 35 mm, about 40 mm, about 45 mm, or about 50 mm to any of about 55 mm, about 60 mm, about 65 mm, about 70 mm, about 75 mm, about 80 mm, about 85 mm, about 90 mm, about 95 mm, or about 100 mm, or any sub-range or single value therein. In one embodiment, the depth of the first container 110 and of the second container 120 range from about 40 mm to about 70 mm. The depth of the first container 110 may be the same or different from the depth of the second container 120.

In certain embodiments, the interior of each container has a fill capacity to accommodate a fill composition volume of up to about 2 ml, up to about 3 ml, up to about 4 ml, up to about 5 ml, up to about 6 ml, up to about 7 ml, up to about 8 ml, up to about 9 ml, up to about 10 ml, up to about 11 ml, up to about 12 ml, up to about 13 ml, up to about 14 ml, or up to about 15 ml, or in sub-range or single value therein. In certain embodiments, the interior of each container is filled up to about 98%, up to about 95%, up to about 90%, up to about 85%, up to about 80%, up to about 75%, up to about 70%, up to about 60%, or up to about 50%, or in sub-range or single value therein, of the respective container's fill capacity. In one embodiment, each container has a fill capacity ranging from about 5 ml to about 8 ml and each container is filled up to about 75% of the fill capacity (e.g., about 3.75 ml to about 6 ml fill composition filled in each container).

Each component of the apparatus may be made of a corresponding material depending, among other factors, on the target properties and desired performance of that component. In certain embodiments, the first container 110, the second container 120, the first cap 140, the second cap 150, the first applicator 160, the second applicator 170, the sleeve 130, the first cap rod 165, the second cap rod 175, the first wiper 180, and the second wiper 190 are made of a material independently selected from acrylonitrile butadiene styrene (ABS), polypropylene (PP), polyethylene (PE), low density polyethylene (LDPE), nylon, polybutylene terephthalate (PBT), styrene acrylonitrile resin (SAN), polyethylene terephthalate glycol (PETG), or a combination thereof.

In certain embodiments, the sleeve 130, the first cap 140, and the second cap 150 are made of a material independently selected from ABS, SAN, PETG, or a combination thereof. In one embodiment, the sleeve 130 is made of ABS. In one embodiment, the sleeve 130 is made of PETG. In one embodiment, the first cap 140 is made of the same material as the second cap 150 and both are made of ABS. In another embodiment, the first cap 140 is made of a different material from second cap 150.

In certain embodiments, the first cap rod 165, the second cap rod 175, the first container 110, and the second container 120 are made of a material independently selected from PP, PE, PETG, or a combination thereof. In one embodiment, cap rod 165 and cap rod 175 are made of the same material. In another embodiment, cap rod 165 and cap rod 175 are made of different materials. In one embodiment, cap rod 165 and cap rod 175 are both made of PP. In one embodiment, the first container 110 is made of the same material as the second container 120 and both are made of PP. In one embodiment, the first container 110 is made of the same material as the second container 120 and both are made of PETG. In another embodiment, the first container 110 is made of a different material from second container 120.

In certain embodiments, the first wiper 180 and the second wiper 190 are made of a material independently selected from rubber (e.g., butadiene-acrylonitrile-rubber (NBR)), LDPE, PP, or a combination thereof. In one embodiment, the first wiper 180 is made of the same material as the second wiper 190 and both are made of LDPE. In one embodiment, the first wiper 180 is made of the same material as the second wiper 190 and both are made of rubber. In another embodiment, the first wiper 180 is made of a different material from second wiper 190. In one embodiment, the first wiper 180 is made of rubber (e.g., when the first container includes a cosmetic nail whitening composition) and the second wiper 190 and is made of LDPE (e.g., when the second container includes an anti-fungal composition).

In certain embodiments, the first applicator 160 and the second applicator 170 are made of a material independently selected from nylon or PBT. In one embodiment, the first applicator 160 is a brush applicator made of PBT. In one embodiment, the second applicator 170 is a felt tip applicator made of nylon.

Method of Preparation

In some embodiments, the instant disclosure is directed to a method of preparing any of the apparatuses described herein. The method includes assembling a first container and a second container in a sleeve. In one embodiment, the first container includes an anti-fungal composition and the second container includes a cosmetic nail whitening composition. In another embodiment, the first container includes a cosmetic nail whitening composition and the second container includes an anti-fungal composition. While the instant method may refer to the contents of the first container and the second container as including an anti-fungal composition and a cosmetic agent composition, such reference should not be construed as limiting. It should be understood by those skilled in the art that other compositions may be suitably filled in the first container and second container and that the apparatus described herein may be useful for other uses besides anti-fungal treatment.

In certain embodiments, an apparatus according to the instant disclosure is prepared by assembling a first container 110 and a second container 120 in a sleeve 130. In the depicted embodiment, the first container 110 has a first bottom closed end 112, a first open top end 114 opposite from the first bottom closed end 112, and a first wall 116 extending from the first bottom closed end 112 (through a first neck portion 118) to the first top open end 114. Similarly, the second container 120 has a second bottom closed end 122, a second open top end 124 opposite from the second bottom closed end 122, and a second wall 126 extending from the second bottom closed end 122 (through a second neck portion 128) to the second top open end 124. In the depicted embodiment, the first container 110 and the second container 120 are assembled in the sleeve 130 such that the first bottom closed end 112 is proximate to the second bottom closed end 122 and the first top open end 114 faces an opposite direction from the second top open end 124. Further, in the depicted embodiment, the sleeve 130 extends along the first wall 116 and the second wall 126.

In certain embodiments, assembling the first container 110 and the second container 120 in the sleeve 130 may be done removably or non-removably. For instance, assembling may include gluing sleeve 130 to the first wall 116 of first container 110 and to the second wall 126 of second container 120, such that the first container 110 and the second container 120 remain in place and are not removable from sleeve 130. Alternatively, assembling may include wrapping sleeve 130 around the first wall 116 of first container 110 and to the second wall 126 of second container 120. In another embodiment, assembling may include slipping sleeve 130 onto the first container 110 and the second container 120. In yet another embodiment, assembling may include mechanically coupling sleeve 130 to the first container 110 and the second container 120.

In certain embodiments, assembling may include aligning first container 110 and second container 120 within sleeve 130 and/or connecting first container 110 and second container 120 to each other removably or non-removably. For instance, assembling may include gluing first closed bottom end 112 of first container 110 to the second closed bottom end 122 of second container 120 such that the containers remain connected to each other non-removably. Alternatively, assembling may include mechanically coupling first closed bottom end 112 and second closed bottom end 122 to each other or to an intermediate component within sleeve 130.

In certain embodiments, the method of preparing the apparatus further includes filling the interior of the first container 110, defined by the first closed bottom end 112, the first top open end 114, and the first wall 116 (including the first neck portion 118), with a first composition. Similarly, the method of preparing the apparatus further includes filling the interior of the second container 120, defined by the second closed bottom end 122, the second top open end 124, and the second wall 126 (including the second neck portion 128), with a second composition. In one embodiment, the first composition in the first container includes an anti-fungal active pharmaceutical ingredient and the second composition in the second container includes a cosmetic agent, such as a nail whitener. In another embodiment, the first composition in the first container includes a cosmetic agent, such as a cosmetic nail whitener, and the second composition in the second container includes an anti-fungal active pharmaceutical ingredient.

In certain embodiments, the interior of each container is filled up to about 98%, up to about 95%, up to about 90%, up to about 85%, up to about 80%, up to about 75%, up to about 70%, up to about 60%, or up to about 50%, or in sub-range or single value therein, of the respective container's fill capacity.

In certain embodiments, the method of preparing the apparatus further includes removably closing the first top open end 114 of the first container 110 with a first cap 140. In certain embodiments, prior to closing the first top open end 114 of the first container 110 with the first cap 140, one or more of the following steps may be performed: placing a first wiper 180 onto the first top open end 114 and/or inserting the first applicator 160 and the first stem 166 into the interior of the first container and/or engaging the first neck portion 118 with the first internal cap assembly 168 of the first cap rod assembly 165.

In certain embodiments, the method of preparing the apparatus further includes removably closing the second top open end 124 of the second container 120 with a second cap 150. In certain embodiments, prior to closing the second top open end 124 of the second container 120 with the second cap 150, one or more of the following steps may be performed: placing a second wiper 190 onto the second top open end 124 and/or inserting the second applicator 170 and the second stem 176 into the interior of the second container and/or engaging the second neck portion 128 with the second internal cap assembly 178 of the second cap rod assembly 175.

Method of Treatment

In some embodiments, the instant disclosure is directed to a method of treating nail and/or skin fungus. The method includes applying an anti-fungal composition from a container having the anti-fungal composition onto the skin and/or nail. The method further includes applying a cosmetic nail whitening composition from a different container having the nail whitening composition onto the nail. The container with the anti-fungal composition and the different container with the nail whitening composition may correspond to any of the first container and second containers described herein and are assembled together in a single apparatus, such as any of the apparatuses described hereinbefore. Applying any of the compositions (whether anti-fungal composition or cosmetic agent) can be done with a corresponding applicator (such as the first applicator and the second applicator described hereinbefore).

In some embodiments, the cosmetic nail whitening composition can be a medical device. The term “medical device,” as used herein, refers to a composition that has no pharmacologic action but a physical action. In certain embodiments, a step of applying a cosmetic nail whitening composition to a given area may also refer to applying a medical device.

In some embodiments, the method of treatment may include applying the anti-fungal composition and/or the cosmetic nail whitening composition once daily, twice daily, three times daily, four times daily, five times daily, or six times daily onto a target area (such as a nail or the skin surrounding the nail) of a patient in need thereof.

In certain embodiments, the duration of the treatment may range from about 1 day, about 2 days, about 3 days, or about 4 days, to about 5 days, about 6 days, about 1 week, about 10 days, about two weeks, about three weeks, about four weeks, about 1 month, about 2 months, or about 3 months.

In one embodiment, the method of treatment includes applying the anti-fungal composition and/or the cosmetic nail whitening composition twice daily (e.g., morning and evening) for about four weeks, optionally followed by maintenance via application of the anti-fungal composition and/or the cosmetic nail whitening composition once daily.

Exemplary anti-fungal active pharmaceutical ingredients suitable for the anti-fungal composition include, without limitations, amorolfine, amphotericin B, carbol-fuchsin, ciclopirox, clotrimazole, dapsone econazole, fluconazole, flucytosine, griseofulvin, Gentian Violet, haloprogin, itraconazole, ketoconazole, mafenide, miconazole, miconazolekl, naftifine, nystatin, oxiconazole, silver sulfadiazine, sulconazole terbinafine, tioconazole, tolnafiat, tolnaftate, undecylenic acid, voriconazole, pharmaceutically acceptable salts thereof, and combinations thereof.

Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

The anti-fungal composition and/or the cosmetic agent composition may include additional pharmaceutically acceptable excipients, such as, without limitations, antioxidants, buffering agents, diluents, surfactants, solubilizers, stabilizers, hydrophilic polymers, absorption or permeability enhancers, preservatives, osmotic agents, isotonicity agents, chelating agents, pH adjusting agents, solvents, co-solvents, viscosity agents, gelling agents, suspending agents or combinations thereof.

In one embodiment, exemplary excipients in the anti-fungal compositions described herein, include, without limitations, one or more of: water, glycerin, propylene glycol, DMDM hydantoin (e.g., Glydant), Aloe Vera Gel (e.g., aloe brbadensis gel), ascorbic acid (vitamin C), ethylenediaminetetraacetic acid (EDTA), titanium dioxide, mineral oil, dimethicone (e.g., dimethicone 350), olive oil, tocopheryl acetate (vitamin E), PEG-100 stearate, tea tree oil, lavender oil, eucalyptol, phenoxetol, and xanthan gum.

In one embodiment, exemplary excipients in the cosmetic nail whitening compositions described herein, include, without limitations, one or more of: ethyl lactate, water, glyceroleum, lactic acid, 2′-4′-dihydroxyacetophone, urea, glycerin, hydrogen peroxide, Aloe Vera Gel, lavender oil, eucalyptol, citric acid 50% solution, citric acid anhydrous, and purified water.

In a first embodiment, the instant disclosure is directed to an apparatus comprising: a first container having a first bottom closed end, a first top open end opposite from the first bottom closed end, and a first wall extending from the first bottom closed end to the first top open end; a first cap coupled to a first applicator, the first cap is configured to removably close the first top open end of the first container; a second container having a second bottom closed end, a second top open end opposite from the second bottom closed end, and a second wall extending from the second bottom closed end to the second top open end; a second cap coupled to a second applicator, the second cap is configured to removably close the second top open end of the second container; and a sleeve extending along the first wall and the second wall, wherein the first container and the second container are assembled in the sleeve such that the first bottom closed end is proximate to the second bottom closed end and the first top open end faces an opposite direction from the second top open end.

In a second embodiment, the instant disclosure is directed to the apparatus of the first embodiment, wherein the first bottom closed end, the first top open and, and the first wall, together define a first interior of the first container, and wherein the first interior is filled with a cosmetic ingredient.

In a third embodiment, the instant disclosure is directed to the apparatus of the second embodiment, wherein the cosmetic ingredient is a whitening agent.

In a fourth embodiment, the instant disclosure is directed to the apparatus of any one of the first to third embodiments, wherein the second bottom closed end, the second top open end, and the second wall, together define a second interior of the second container, and wherein the second interior is filled with an active pharmaceutical ingredient.

In a fifth embodiment, the instant disclosure is directed to the apparatus of the fourth embodiment, wherein the active pharmaceutical ingredient is an anti-fungal agent.

In a sixth embodiment, the instant disclosure is directed to the apparatus of any one of the first to the fifth embodiments, wherein the first applicator and the second applicator are independently selected from a brushless tip or a brush tip, and wherein the brushless tip is selected from a felt tip or a sponge.

In a seventh embodiment, the instant disclosure is directed to the apparatus of any one of the first to the sixth embodiments, wherein the first container further comprises a first wiper positioned between the first top open end and the first cap when the first container is closed with the first cap.

In an eighth embodiment, the instant disclosure is directed to the apparatus of any one of the first to the seventh embodiments, wherein the second container further comprises a second wiper positioned between the second top open end and the second cap when the second container is closed with the second cap.

In a ninth embodiment, the instant disclosure is directed to the apparatus of any one of the first to the eighth embodiments, wherein the first cap is coupled to the first applicator via a first cap rod.

In a tenth embodiment, the instant disclosure is directed to the apparatus of any one of the first to the ninth embodiments, wherein the second cap is coupled to the second applicator via a second cap rod.

In an eleventh embodiment, the instant disclosure is directed to the apparatus of any one of the first to the tenth embodiments, wherein the first bottom closed end of the first container is attached non-removably to the second bottom closed end of the second container in the sleeve.

In a twelfth embodiment, the instant disclosure is directed to the apparatus of the eleventh embodiment, wherein the first bottom closed end of the first container is glued to the second bottom closed end of the second container.

In a thirteenth embodiment, the instant disclosure is directed to the apparatus of any one of the first to the twelfth embodiments, wherein the sleeve is glued to the first wall and to the second wall.

In a fourteenth embodiment, the instant disclosure is directed to the apparatus of any one of the first to the thirteenth embodiments, wherein the first cap is configured to removably close the first top open end of the first container via a screw on mechanism.

In a fifteenth embodiment, the instant disclosure is directed to the apparatus of any one of the first to the fourteenth embodiments, wherein the second cap is configured to removably close the second top open end of the second container via a screw on mechanism.

In a sixteenth embodiment, the instant disclosure is directed the apparatus of any one of the first to the fifteenth embodiments, wherein the apparatus is airtight when the first container is closed with the first cap and when the second container is closed with the second cap.

In a seventeenth embodiment, the instant disclosure is directed to the apparatus of any one of the first to the sixteenth embodiments, wherein at least one of the first cap and the second cap are ribbed.

In an eighteenth embodiment, the instant disclosure is directed to the apparatus of any one of the first to the seventeenth embodiments, wherein the first container, the second container, the first cap, the second cap, the first applicator, the second applicator, the sleeve, the first cap rod, the second cap rod, the first wiper, and the second wiper are made of a material independently selected from acrylonitrile butadiene styrene (ABS), polypropylene (PP), polyethylene (PE), low density polyethylene (LDPE), nylon, polybutylene terephthalate (PBT), styrene acrylonitrile resin (SAN), polyethylene terephthalate glycol (PETG), rubber, or a combination thereof.

In a nineteenth embodiment, the instant disclosure is directed to the apparatus of the eighteenth embodiment, wherein the first cap, the second, and the sleeve are made of ABS, SAN, PETG, or a combination thereof.

In a twentieth embodiment, the instant disclosure is directed to the apparatus of any one of the eighteenth to the nineteenth embodiments, wherein the first cap rod, the second cap rod, the first container, and the second container are made of PP, PE, PETG, or a combination thereof.

In a twenty first embodiment, the instant disclosure is directed to the apparatus of any one of the eighteenth to the twentieth embodiments, wherein the first wiper and the second wiper are made of LDPE, PP, rubber, or a combination thereof.

In a twenty second embodiment, the instant disclosure is directed to the apparatus of any one of the eighteenth to the twenty first embodiments, wherein the first applicator and the second applicator are made of a material independently selected from nylon and PBT.

In a twenty third embodiment, the instant disclosure is directed to a method of treating nail fungus comprising: applying a cosmetic agent or medical device with a first applicator on a nail; and applying an anti-fungal active pharmaceutical ingredient with a second applicator on a skin around the nail and between toes, wherein the first applicator and the second applicator are part of an apparatus comprising: a first container having a first bottom closed end, a first top open end opposite from the first bottom closed end, and a first wall extending from the first bottom closed end to the first top open end, wherein the first bottom closed end, the first top open and, and the first wall, together define a first interior of the first container, and wherein the first interior is filled with the cosmetic agent; a first cap coupled to the first applicator, the first cap removably closing the first top open end of the first container; a second container having a second bottom closed end, a second top open end opposite from the second bottom closed end, and a second wall extending from the second bottom closed end to the second top open end, wherein the second bottom closed end, the second top open end, and the second wall, together define a second interior of the second container, and wherein the second interior is filled with the anti-fungal active pharmaceutical ingredient; a second cap coupled to the second applicator, the second cap removably closing the second top open end of the second container; and a sleeve extending along the first wall and the second wall, wherein the first container and the second container are assembled in the sleeve such that the first bottom closed end is proximate to the second bottom closed end and the first top open end faces an opposite direction from the second top open end.

In a twenty fourth embodiment, the instant disclosure is directed to a method of preparing an apparatus, comprising: assembling a first container and a second container in a sleeve, wherein the first container has a first bottom closed end, a first top open end opposite from the first bottom closed end, and a first wall extending from the first bottom closed end to the first top open end, wherein the second container has a second bottom closed end, a second top open end opposite from the second bottom closed end, and a second wall extending from the second bottom closed end to the second top open end, wherein the sleeve extends along the first wall and the second wall, wherein the first container and the second container are assembled in the sleeve such that the first bottom closed end is proximate to the second bottom closed end and the first top open end faces an opposite direction from the second top open end.

In a twenty fifth embodiment, the instant disclosure is directed to the method of the twenty fourth embodiment, further comprising filling cosmetic agent into the first interior of a first container, the first interior being defined by the first bottom closed end, the first top open end, and the first wall.

In a twenty sixth embodiment, the instant disclosure is directed to the method of any one of the twenty fourth to the twenty fifth embodiments, further comprising filling an anti-fungal active pharmaceutical ingredient into a second interior of the second container, the second interior being defined by the second bottom closed end, the second top open end, and the second wall.

In a twenty seventh embodiment, the instant disclosure is directed to the method of any one of the twenty fourth to the twenty sixth embodiments, wherein assembling comprises gluing the first bottom closed end of the first container to the second bottom closed end of the second container.

In a twenty eighth embodiment, the instant disclosure is directed to the method of any one of the twenty fourth to the twenty seventh embodiments, wherein assembling comprises gluing the sleeve to the first wall and to the second wall.

In a twenty ninth embodiment, the instant disclosure is directed to the method of any one of the twenty fourth to the twenty eighth embodiments, further comprising removably closing the first top open end of the first container with a first cap.

In a thirtieth embodiment, the instant disclosure is directed to the method of any one of the twenty fourth to the twenty ninth embodiments, further comprising removably closing the second top open end of the second container with a second cap.

In a thirty first embodiment, the instant disclosure is directed to an apparatus comprising: a first container having a whitening composition; and a second container having an anti-fugal composition.

In a thirty second embodiment, the instant disclosure is directed to a method of treating nail fungus comprising: applying a whitening composition from a first container having the whitening composition onto a nail; and applying an anti-fungal composition from a second container having the anti-fungal composition on the skin around the nail and in between toes, wherein the first container and the second container are assembled together in the same apparatus.

In a thirty third embodiment, the instant disclosure is directed to a method of preparing an apparatus comprising: assembling a first container having a whitening composition and a second container having an antifungal composition in a sleeve.

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The disclosure has been described with reference to specific exemplary embodiments thereof. The specification and drawings are, accordingly, to be regarded in an illustrative rather than a restrictive sense. Various modifications of the disclosure in addition to those shown and described herein will become apparent to those skilled in the art and are intended to fall within the scope of the appended claims.

What is claimed is:

1. An apparatus comprising:
a first container having a first bottom closed end, a first top open end opposite from the first bottom closed end, and a first wall extending from the first bottom closed end to the first top open end;
a first cap coupled to a first applicator, the first cap is configured to removably close the first top open end of the first container;
a second container having a second bottom closed end, a second top open end opposite from the second bottom closed end, and a second wall extending from the second bottom closed end to the second top open end;
a second cap coupled to a second applicator, the second cap is configured to removably close the second top open end of the second container; and
a sleeve extending along the first wall and the second wall,
wherein the first container and the second container are assembled in the sleeve such that the first bottom closed end is proximate to the second bottom closed end and the first top open end faces an opposite direction from the second top open end.

2. The apparatus of claim 1, wherein the first bottom closed end, the first top open and, and the first wall, together define a first interior of the first container, and wherein the first interior is filled with a cosmetic ingredient.

3. The apparatus of claim 2, wherein the cosmetic ingredient is a whitening agent.

4. The apparatus of claim 1, wherein the second bottom closed end, the second top open end, and the second wall, together define a second interior of the second container, and wherein the second interior is filled with an active pharmaceutical ingredient.

5. The apparatus of claim 4, wherein the active pharmaceutical ingredient is an anti-fungal agent.

6. The apparatus of claim 1, wherein the first applicator and the second applicator are independently selected from a brushless tip or a brush tip, and wherein the brushless tip is selected from a felt tip or a sponge.

7. The apparatus of claim 1, wherein the first container further comprises a first wiper positioned between the first top open end and the first cap when the first container is closed with the first cap, and wherein the second container further comprises a second wiper positioned between the second top open end and the second cap when the second container is closed with the second cap.

8. The apparatus of claim 1, wherein the first cap is coupled to the first applicator via a first cap rod, and wherein the second cap is coupled to the second applicator via a second cap rod.

9. The apparatus of claim 1, wherein the first bottom closed end of the first container is attached non-removably to the second bottom closed end of the second container in the sleeve.

10. The apparatus of claim 9, wherein the first bottom closed end of the first container is glued to the second bottom closed end of the second container.

11. The apparatus of claim 1, wherein the sleeve is glued to the first wall and to the second wall.

12. The apparatus of claim 1, wherein the first cap is configured to removably close the first top open end of the first container via a screw on mechanism, and wherein the second cap is configured to removably close the second top open end of the second container via a screw on mechanism.

13. The apparatus of claim 1, wherein the apparatus is airtight when the first container is closed with the first cap and when the second container is closed with the second cap.

14. The apparatus of claim 1, wherein at least one of the first cap and the second cap are ribbed.

15. The apparatus of claim 1, wherein the first container, the second container, the first cap, the second cap, the first applicator, the second applicator, the sleeve, the first cap rod, the second cap rod, the first wiper, and the second wiper are made of a material independently selected from acrylonitrile butadiene styrene (ABS), polypropylene (PP), polyethylene (PE), low density polyethylene (LDPE), nylon, polybutylene terephthalate (PBT), styrene acrylonitrile resin (SAN), polyethylene terephthalate glycol (PETG), rubber, or a combination thereof.

16. The apparatus of claim 15, wherein the first cap, the second, and the sleeve are made of ABS, SAN, PETG, or a combination thereof,
wherein the first cap rod, the second cap rod, the first container, and the second container are made of PP, PE, PETG, or a combination thereof,
wherein the first wiper and the second wiper are made of LDPE, PP, rubber, or a combination thereof, and
wherein the first applicator and the second applicator are made of a material independently selected from nylon and PBT.

17. A method of treating nail fungus comprising:
applying a cosmetic agent or medical device with a first applicator on a nail; and
applying an anti-fungal active pharmaceutical ingredient with a second applicator on a skin around the nail and between toes,
wherein the first applicator and the second applicator are part of an apparatus comprising:
a first container having a first bottom closed end, a first top open end opposite from the first bottom closed end, and a first wall extending from the first bottom closed end to the first top open end, wherein the first bottom closed end, the first top open and, and the first wall, together define a first interior of the first container, and wherein the first interior is filled with the cosmetic agent;
a first cap coupled to the first applicator, the first cap removably closing the first top open end of the first container;
a second container having a second bottom closed end, a second top open end opposite from the second bottom closed end, and a second wall extending from the second bottom closed end to the second top open end, wherein the second bottom closed end, the second top open end, and the second wall, together define a second interior of the second container, and wherein the second interior is filled with the anti-fungal active pharmaceutical ingredient;
a second cap coupled to the second applicator, the second cap removably closing the second top open end of the second container; and
a sleeve extending along the first wall and the second wall,
wherein the first container and the second container are assembled in the sleeve such that the first bottom closed end is proximate to the second bottom closed end and the first top open end faces an opposite direction from the second top open end.

18. A method of preparing an apparatus, comprising:
assembling a first container and a second container in a sleeve,
wherein the first container has a first bottom closed end, a first top open end opposite from the first bottom closed end, and a first wall extending from the first bottom closed end to the first top open end,
wherein the second container has a second bottom closed end, a second top open end opposite from the second bottom closed end, and a second wall extending from the second bottom closed end to the second top open end,
wherein the sleeve extends along the first wall and the second wall,
wherein the first container and the second container are assembled in the sleeve such that the first bottom closed end is proximate to the second bottom closed end and the first top open end faces an opposite direction from the second top open end.

19. The method of claim 18, further comprising one or more of:
filling cosmetic agent into the first interior of a first container, the first interior being defined by the first bottom closed end, the first top open end, and the first wall, filling an anti-fungal active pharmaceutical ingredient into a second interior of the second container, the second interior being defined by the second bottom closed end, the second top open end, and the second wall,
removably closing the first top open end of the first container with a first cap, or removably closing the second top open end of the second container with a second cap.

20. The method of claim 18, wherein assembling comprises one or more of:
gluing the first bottom closed end of the first container to the second bottom closed end of the second container, or
gluing the sleeve to the first wall and to the second wall.
